(19) United States
(12) Patent Application Publication
(10) Pub. No.: US 2025/0257023 A1
(43) Pub. Date: Aug. 14, 2025

(54) SYSTEMS AND METHODS FOR USING GLYCEROL-BASED SCALE INHIBITORS IN WASTE STREAMS

(71) Applicant: Cameron International Corporation, Houston, TX (US)

(72) Inventors: Alyn Jenkins, Aberdeen (GB); James McRae, Crawley (GB); Tore Nordvik, Stavanger (NO); Bianca Covarrubias, Houston, TX (US); Paul Barnes, Aberdeen (GB)

(21) Appl. No.: 18/436,557

(22) Filed: Feb. 8, 2024

(51) Int. Cl. C07C 29/94

(52) U.S. Cl. CPC C07C 29/94

(57) ABSTRACT

Provided herein is a method for generating a glycerol-based scale inhibitor and utilizing it to generate a glycerol-based scale inhibitor solution. The method includes providing a glycerol feed and a scale-inhibiting precursor to generate the glycerol-based scale inhibitor. Further, the method includes providing one or more additives to the one or more glycerol-based scale inhibitor, thereby generating a glycerol-based scale inhibitor solution.

### BACKGROUND

This disclosure relates to a glycerol-based scale inhibitor with one or more functional groups. In particular, the processes may include using a feedstock of glycerol with a scale-inhibiting precursor. The disclosed glycerol-based scale inhibitor is especially useful for anti-scaling applications, such as oil and gas, water treatment, power plants, or geothermal applications.

Low, moderate, or high temperature processes are performed by many industries, such as oil and gas, water treatment, power plant, or geothermal. Such processes are often impacted by scaling depositions. For example, the process for generating hydrocarbons from subterranean reservoirs subjects downhole equipment to harsh conditions, such as high temperatures, caustic chemicals, and limited space. These harsh operating conditions may promote scaling, resulting in poor performance and function of the downhole equipment and an overall reduction in productivity.

To this end, anti-scaling chemistry is ubiquitous within the oilfield chemistry for the mitigation of inorganic scale. The materials utilized in the development of the scaling agents may be sourced from petroleum refineries and are further functionalized to generate a desired product. Due to the process by which the base materials for the traditional scaling agents are sourced, certain scaling agents exhibit a significant carbon footprint in their manufacturing process. Therefore, current limitations in the production of scaling agents include finding sustainable, environmentally friendly base materials and methods. Moreover, limitations in sustainable, environmentally friendly base materials and methods for phosphonate-based chemistry are hindering a further decrease in carbon footprint in various fields such as oil and gas, water treatment, power plant, or geothermal. It may be desirable to develop more efficient base materials and methods for accessing sustainable, environmentally friendly scaling agents to facilitate a decrease in carbon footprint while mitigating inorganic scale.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In certain embodiments, the present disclosure relates to a method for generating a glycerol-based scale inhibitor solution. The method includes providing a glycerol feed and a scale-inhibiting precursor feed. The method also includes generating one or more glycerol-based scale inhibitors by contacting the glycerol feed with the scale-inhibiting precursor feed. Further, the method includes providing the glycerol-based scale inhibitor feed and one or more additives to generate a glycerol-based scale inhibitor solution.

In certain embodiments, a composition includes a glycerol-based scale inhibitor. The glycerol-based scale inhibitor includes a glycerol-analogue core and one or more chelating groups linked to the glycerol-analogue core via one or more ethers of the glycerol-analogue core.

In certain embodiments, a method includes providing a glycerol-based scale inhibitor solution and providing a liquid volume. The liquid volume includes metal ions. The method also includes sequestering the metal ions using the glycerol-based scale inhibitor to form a metal-ion complex. Further, the method includes separating the metal-ion complex and glycerol-based scale inhibitor solution from the liquid volume.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

To assist those of ordinary skill in the relevant art in making and using the subject matter hereof, reference is made to the appended drawings, wherein:

FIG. 1 is a flow diagram of a method for generating a glycerol-based scale inhibitor solution, in accordance with present disclosure;

FIG. 2 illustrates an example of a molecular structure for the glycerol-based scale inhibitor, in accordance with the present disclosure:

FIG. 3A illustrates an example of a glycerol-based scale inhibitor generated with a 1:1 stoichiometric ratio of glycerol:scale-inhibiting precursor, in accordance with the present disclosure;

FIG. 3B illustrate an example of a glycerol-based scale inhibitor generated with a 1:2 stoichiometric ratio glycerol:scale-inhibiting precursor, in accordance with the present disclosure;

FIG. 3C illustrate an example of a glycerol-based scale inhibitor generated with a 1:3 stoichiometric ratio glycerol:scale-inhibiting precursor, in accordance with the present disclosure;

FIG. 4A illustrates an example of a glycerol-based scale inhibitor generated with a 1:1 stoichiometric ratio of glycerol:scale-inhibiting precursor, in accordance with the present disclosure;

FIG. 4B illustrate an example of a glycerol-based scale inhibitor generated with a 1:2 stoichiometric ratio glycerol:scale-inhibiting precursor, in accordance with the present disclosure;

FIG. 4C illustrate an example of a glycerol-based scale inhibitor generated with a 1:3 stoichiometric ratio glycerol:scale-inhibiting precursor, in accordance with the present disclosure;

FIG. 5 is a flow diagram of an example method for using a glycerol-based scale inhibitor solution for sequestering metal ions, in accordance with the present disclosure; and

FIG. 6 illustrates a schematic view of a subsea system where the glycerol-based scale inhibitor solution may be employed in mitigating inorganic scale in oil and gas production.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Furthermore, when introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment,” “an embodiment,” or “some embodiments” of the present disclosure are not intended to be interpreted as excluding the existence of additional embodiments that also incorporate the recited features. Furthermore, the phrase A “based on” B is intended to mean that A is at least partially based on B. Moreover, unless expressly stated otherwise, the term “or” is intended to be inclusive (e.g., logical OR) and not exclusive (e.g., logical XOR). In other words, the phrase A “or” B is intended to mean A, B, or both A and B. elements. In general, it should be noted that “bind,” “sequester,” and/or “chelate” may be used herein interchangeably. All numerical values within the detailed description herein are modified by “about” the indicated value, and take into account experimental error and variations that would be expected by a person having ordinary skill in the art. For example, “about” or “approximately” may refer to ±0.5%, ±1%, ±2, ±5%, ±10%, or ±15%.

As described herein, the term “glycerol” refers to “propane-1,2,3-triol”, also called glycerine, or glycerin. The term “glycerol-based scale inhibitor” refers to a glycerol molecule, but one or more of the hydroxyl functional groups of the original glycerol molecule are functionalized with a scale inhibitor. In general, the “scale inhibitor” may inhibit or prevent scaling by chelating or binding or sequestering positive inorganic cations associated with scale. The term “glycerol-based scale inhibitor solution” refers to a solution that includes the glycerol-based scale inhibitor and additives. The term “glycerol-analogue core” refers to glycerol and its three hydroxyl functional groups that can be functionalized using scale-inhibiting precursors (i.e., thereby forming scale-inhibiting functional groups). The term “carbon backbone” or “main carbon chain” or “backbone” refers to the longest carbon chain in the molecule of the compound or group in question.

The term “scale-inhibiting precursor” or “chelating precursor” refers to a molecule that includes functional groups that can bind to positively charged metal ions. For example, a molecule including phosphate groups, phosphonate groups, sulfonate groups, sulfate groups, phosphate ester groups, carboxylic acid groups, another other functional group, or a combination thereof, is a scale-inhibiting precursor.

The term “additive” refers to any other molecule, solvent, or buffer that may be added to the glycerol-based scale inhibitor to generate the glycerol-based scale inhibitor solution. The term “buffer” or “buffering agent” refers to solutions that can resist changes in pH when acidic or basic solutions are added.

A scale inhibitor solution made with sustainable base materials may provide satisfactory chelating properties to positively charged metal ions in industrial applications in which existing scale inhibitors may be unsuitable due to their significant carbon footprint. However, efficient syntheses for scale inhibitor solutions for anti-scaling applications synthesized with sustainable, environmentally friendly base materials having an acceptable combination of solubility and chelating abilities have yet to be identified.

This disclosure relates to techniques for generating, producing, or otherwise performing one or more reactions to yield a glycerol-based scale inhibitor as a sustainable, environmentally friendly material that may be used in oil and gas operations that has advantageous chelating and solubility properties. Biodiesel waste streams containing crude glycerol are a suitable source for glycerol, acting as a sustainable base material. As such, crude glycerol containing waste streams can be functionalized with appropriate functional groups to form the glycerol-based scale inhibitor that prevent scale depositions by binding to inorganic cations. In general, the techniques discussed herein include generating the glycerol-based scale inhibitor by reacting glycerol (i.e., propane-1,2,3-triol) with one or more scaling precursors (e.g., different or the same scale-inhibiting precursors). The stoichiometric ratio of the one or more scale-inhibiting precursors to the glycerol may be between approximately 1 to 3, thereby generating a glycerol-based scale inhibitor having between 1 to 3 scale-inhibiting functional groups. In certain embodiments, it may be advantageous to generate the disclosed conditions including one or more of a glycerol feed, a scale-inhibiting precursor feed, a type of scale-inhibiting precursor, a stoichiometric equivalent addition of scale-inhibiting precursor, or a combination thereof, to obtain a glycerol-based scale inhibitor. The disclosed techniques provide a glycerol-based scale inhibitor with tunable activity (e.g., chelating activity, scaling activity) by varying the stoichiometric ratio of the one or more scale-inhibiting precursors, utilizing a type of scale-inhibiting precursor, or utilizing a combination of scale-inhibiting precursors. As referred to herein, “chelating activity” refers to the ability of a scale inhibitor to bind ions (e.g., positive ions in a solution). For example, the “chelating activity” may refer to the number of ions a single scale inhibitor is capable of binding and/or the binding affinity of the scale inhibitor for binding the ions. In some embodiments, the techniques include generating a glycerol-based scale inhibitor solution using the glycerol-based scale inhibitor. In certain embodiments, generating the glycerol-based scale inhibitor includes providing additives (e.g., other molecules, buffers, or solvents) to the glycerol-based scale inhibitor. It is presently recognized that such additives may provide tuning of the chelating activity of the glycerol-based scale inhibitor in the glycerol-based scale inhibitor solution (e.g., adjusting the pH of the scale-inhibiting functional groups of the glycerol-based scale inhibitor). In any case, the glycerol-based scale inhibitor solution may be provided to a liquid volume (e.g., a subterranean reservoir, conduits in a downhole tool, or fluid volumes where tools operate in the presence of a relatively high concentration of metal ions) to chelate, sequester, bind, or otherwise coordinate to metal ions, thereby forming a metal ion complex and reducing or preventing scaling. In any case, the disclosed glycerol-based scale inhibitor may be used to generate glycerol-based scale inhibitor solutions for use as sustainably-synthesized scale inhibitors in industrial applications, which may be more efficient and sustainable as compared to existing scale inhibitor fluids. Further, the disclosed glycerol-based scale inhibitor, and the glycerol-based scale inhibitor solution, have a chelating activity that is readily tunable by way of the stoichiometric ratio of scale-inhibiting precursors used to generate the glycerol-based scale inhibitor.

In certain embodiments, a method includes reacting a glycerol feed with a scale-inhibiting precursor feed to generate one or more glycerol-based scale inhibitors by contacting the glycerol feed with the scale-inhibiting precursor. The scale-inhibiting precursor may include phosphates, sulfonates, sulfates, carboxylic acids, alkenyl phosphonic acids, phosphonates, phosphate esters, or any combination thereof. In some embodiments, additives may be added to the glycerol-based scale inhibitor to generate a glycerol-based scale inhibitor solution. The additives may include other molecules, solvents, or buffers. The solvents may include phosphonates, sulfonates, sulfates, phosphate esters, phosphates, alkenyl phosphonic acids, glycols, water, alcohols, carboxylic acids, protic solvents, solvents miscible with water, or a combination thereof. In some embodiments, the buffering agents may include phosphate-based buffers, hydroxide-based buffers, carbonate-based buffers, bicarbonate-based buffers, amine-based buffers, acetate-based buffers, or any combination thereof.

In certain embodiments, a composition of the glycerol-based scale inhibitor includes a glycerol-analogue core and one or more chelating groups linked to the glycerol analogue core via one or more ethers of the glycerol analogue core. In some embodiments, the glycerol analogue core may include glycerol or analogues of glycerol, such as propane-1,2,3-triamine or 1,2,3-trimercaptopropane. In some embodiments, the glycerol analogue core may include three or less hydroxyl functional groups. In some embodiments, the chelating group may include phosphonates, alkenyl phosphonic acids, phosphates, phosphate esters, or any combination thereof.

In certain embodiments, a method includes providing a glycerol-based scale inhibitor solution to a liquid volume that includes metal ions. The metal ions are sequestered by the glycerol-based scale inhibitor, forming a metal-ion complex. The metal-ion complex and glycerol-based scale inhibitor are separated from the liquid volume. In general, the glycerol-based scale inhibitor solution may be utilized in mitigating inorganic scaling in industrial applications. In certain embodiments, the metal ions may include metal carbonates, metal sulfates, metal hydroxides, metal sulfides, zinc sulfides, lead sulfides, or any combination thereof. In some embodiments, the glycerol-based scale inhibitor solution is injected via a subsea fluid injection system into a subterranean reservoir to mitigate inorganic scale.

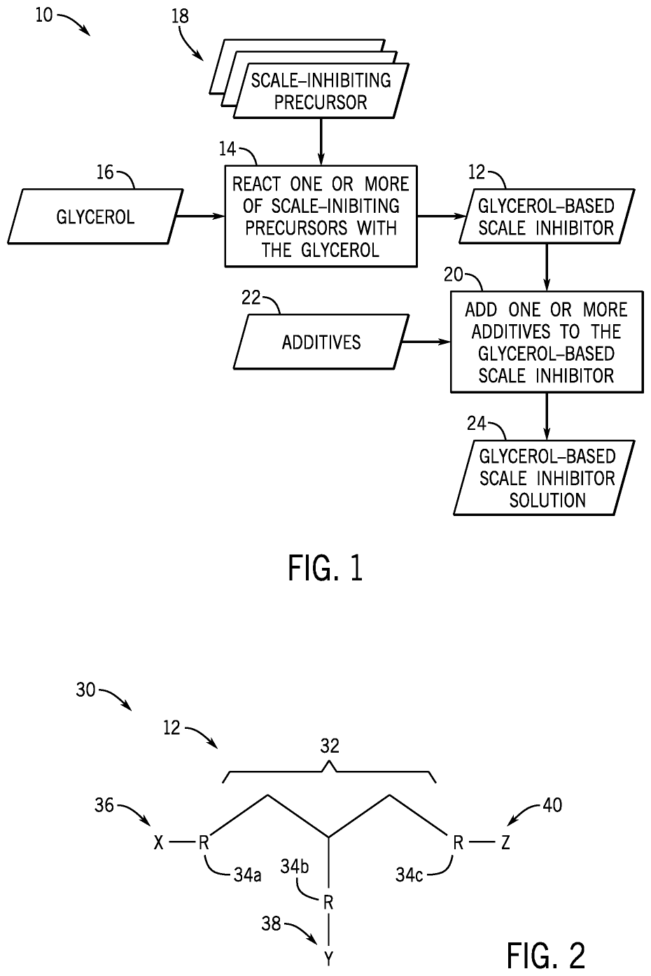

With the foregoing in mind, FIG. 1 illustrates a flow diagram of a method 10 for generating the glycerol-based scale inhibitor solution 24 in accordance with certain embodiments of the present disclosure. In general, the method 10, at block 14, includes reacting a glycerol 16 (e.g., glycerol feed) and a scale-inhibiting precursor 18 (e.g., scale-inhibiting precursor feed) in a reactor, generating a glycerol-based scale inhibitor 12. Further, the method includes 10, at block 20, providing additives 22 to the glycerol-based scale inhibitor 12, thereby generating a glycerol-based scale inhibitor solution 24. As described herein, the glycerol-based scale inhibitor solution 24 may include advantageous properties such as tunable chelating abilities and solubility properties. Block 14 and block 20 are discussed in more detail below.

Referring to the method 10, at block 14, the glycerol-based scale inhibitor 12 may be generated by reacting the glycerol 16 and scale-inhibiting precursor 18 in a solvent. In some embodiments, reacting the glycerol 16 and scale-inhibiting precursor 18 may include providing a feed (e.g., glycerol feed and scale-inhibiting precursor feed) including the glycerol 16 and scale-inhibiting precursor 18 in the presence of a solvent into a reaction vessel to generate the glycerol-based scale inhibitor 12. For example, the solvent may serve as a reaction medium and may include phosphonates, sulfonates, sulfates, phosphate esters, phosphates, alkenyl phosphonic acids, carboxylic acids, glycols, water, alcohols, protic solvents, solvents miscible with water, or a combination thereof.

The glycerol 16 includes three hydroxyl functional groups. In some embodiments, it may be advantageous to convert all three hydroxyl functional groups into a new scale-inhibiting functional group. In some embodiments, it may be advantageous to convert only one or two of the hydroxyl functional groups into a new scale-inhibiting functional group. To do so, the stoichiometric ratio of the scale-inhibiting precursor 18 to the glycerol 16 may be from one to three. For example, one of the hydroxyl groups in the glycerol 16 can react with one scale-inhibiting precursor 18, two of the hydroxyl groups in the glycerol 16 can react with two scale-inhibiting precursors 18, or three of the hydroxyl groups in the glycerol 16 can react with three scale-inhibiting precursor 18. While the description herein relates to the glycerol 16, it should be noted that the above described techniques may include glycerol analogues having one or more thiols or amines instead of the one or more hydroxyl groups of glycerol. For example, the above techniques may utilize propane-1,2,3-triamine or 1,2,3-trimercaptopropane.

In some embodiments, the scale-inhibiting precursor 18 is generally a molecule with a functional group or multiple functional groups that is capable of binding to positively-charged metal ions. In one embodiment, the scale-inhibiting precursor 18 may be a phosphonate (e.g., C—PO(OR)₂, where R may be an alkenyl group, an aryl group, alkyl group, hydrogen, or any combination thereof), alkenyl phosphonic acid (e.g., vinyl phosphonic acid, alternative phosphonic acids with suitably functionalized double bonds in which the double bond is in conjugation with an electron withdrawing group (e.g., carbonyl (C═O), forming a suitable Michael acceptor, salts of phosphonic acids (e.g., sodium phosphonates, potassium phosphonates, etc.)). For example, in an embodiment when the scale-inhibiting precursor 18 include alkenyl phosphonic acids, the scale-inhibiting precursor 18 may include one or more types of alkenyl phosphonic acid, such as vinyl phosphonic acids (e.g., a first type of phosphonic acid) or alternative phosphonic acids with suitably functionalized double bonds in which the double bond is in conjugation with an electron withdrawing group, forming a suitable Michael acceptor. In another embodiment, phosphorus oxychloride (POCl₃) may be utilized as the scale-inhibiting precursor 18 for the formation of phosphate esters (e.g., O═P(OR)₃, where R may be an alkenyl group, an aryl group, alkyl group, hydrogen, or any combination thereof). In some embodiments, the scale-inhibiting precursor 18 may include functional groups that may be phosphates, phosphonates, or alkenyl phosphonic acids that include one or more oxides and/or one or more hydroxides that bind to a positive metal ion.

In another embodiment, the scale-inhibiting precursor 18 may include functionalized carboxylic acids (e.g., acrylic acid, maleic acid, anhydride, itaconic acid, etc.) as suitable Michael acceptors. In a further embodiment, the scale-inhibiting precursor 18 may be sulfonates (e.g., (R—(═S)₂—O—), where R may be an alkenyl group, an aryl group, alkyl group, hydrogen, or any combination thereof), vinyl sulfonic acid, chlorosulfuric acid, salts of sulfonic acids (e.g., sodium sulfonate, potassium sulfonate, etc.). In general, it should be noted that the scale-inhibiting precursor 18 may include a combination of precursors such as phosphate groups, phosphonate groups, sulfonate groups, sulfate groups, phosphate ester groups, carboxylic acid groups, another other functional group, or a combination thereof.

As discussed above with respect to block 20, the resulting glycerol-based scale inhibitor 12 may be combined with one or more additives 22 to generate the glycerol-based scale inhibitor solution 24. For example, the glycerol-based scale inhibitor 12 may be provided to a reaction vessel (e.g., a glycerol-based scale inhibitor feed) with additives 22 (e.g., additive feed) to generate the glycerol-based scale inhibitor solution 24. As such, the glycerol-based scale inhibitor solution 24 may include additives 22, such as other molecules, solvents, or buffers. The buffers adjust the pH of the glycerol-based scale inhibitor solution 24, freeing up the number of binding groups available within the glycerol-based scale inhibitor 12, allowing the glycerol-based scale inhibitor 12 to bind to positively charged metal ions. As such, the additives 22 may include buffering agents, such as phosphate-based buffers, hydroxide-based buffers, carbonate-based buffers, bicarbonate-based buffers, amine-based buffers, acetate-based buffers, or any combination thereof. Further, solvents may be provided as an additive 22 during the production of glycerol-based scale inhibitor solution 24. The solvent may serve as a reaction medium and may include phosphonates, sulfonates, sulfates, phosphate esters, phosphates, alkenyl phosphonic acids, glycols, water, alcohols, carboxylic acids, protic solvents, solvents miscible with water, or a combination thereof.

As described herein, the glycerol 16 is reacted with the scale-inhibiting precursor 18 to generate the glycerol-based scale inhibitor 12. Additives 22 are provided to the glycerol-based scale inhibitor 12 to generate the glycerol-based scale inhibitor solution 24. As such, the glycerol-based scale inhibitor solution can be utilized for anti-scaling applications due to its advantageous synthesis with sustainable base materials, such as glycerol, providing tunable chelating and solubility properties. With the preceding in mind, FIG. 2 illustrates an example of a molecular structure 30 for the glycerol-based scale inhibitor 12 in accordance with certain embodiments of the present disclosure. In general, the glycerol-based scale inhibitor 12 includes the main carbon chain 32 of the glycerol 16. As such, the main carbon chain 32 serves as the backbone of the glycerol-based scale inhibitor 12 or glycerol analogue.

The glycerol-based scale inhibitor 12 also includes R groups 34a, 34b, and 34c (e.g., collectively, R groups 34). The R groups 34a, 34b, and 34c may be any oxygen (O), sulfur (S), or nitrogen (N) atom within glycerol (e.g., O atoms) or glycerol analogues (e.g., S or N atoms) that may be used in certain embodiments. Further, the glycerol-based scale inhibitor 12 includes scale-inhibiting functional groups, which are indicated by X, Y, and Z groups 36, 38, and 40. In general, the scale-inhibiting functional groups or X, Y, and Z groups 36, 38, and 40 are the scale-inhibiting precursors 18 that are bonded to the main carbon chain 32 of the glycerol-based scale inhibitor 12. For example, the scale-inhibiting functional group may be linked to the glycerol-based scale inhibitor 12 via an ether in an embodiment when the R group 34 is an O atom. As referred to herein, “linked” refers to covalently bonded. It should be noted that the scale-inhibiting precursor 18 refers to a molecule that has not formed a bond with glycerol 16, whereas the scale-inhibiting functional group has formed a bond with glycerol 16, generating the glycerol-based scale inhibitor 12. As described herein, the glycerol-based scale inhibitor solution 24 may include advantageous properties, such as tunable chelating ability and solubility properties.

Referring to FIG. 2, the main carbon chain 32 is the carbon backbone within the glycerol-based scale inhibitor 12. In certain embodiments, a different base material with a longer backbone including 3 hydroxyl functional groups may be utilized (e.g., pentane-1,3,5-triol, heptane-1,4,7-triol, etc.). As described herein, R groups 34 may be an O atom within the hydroxyl functional group of glycerol. In some embodiments, R groups 34 include an S atom within a glycerol analogue, such as 1,2,3-trimercaptopropane. In some embodiments, R groups 34 may be an N atom within a glycerol analogue, such as propane-1,2,3-triamine. In some embodiments, R groups 34 may include all O atoms, all S atoms, all N atoms, or any combination thereof.

In general, it should be noted that X, Y, and Z groups 36, 38, and 40 may represent an atom within the scale-inhibiting precursor 18 that is bonded to R group 34 of the glycerol 16 or glycerol analogues. In some embodiments, X, Y, and Z groups 36, 38, and 40 may represent the scale-inhibiting functional group in its entirety that is bonded to R group 34 of the glycerol 16 or glycerol analogues. In some embodiments, if a scale-inhibiting precursor 18 does not functionalize with the glycerol 16 or glycerol analogues, X, Y, and Z groups 36, 38, and 40 may be a hydrogen (H) atom bonded to O atom. In some embodiments, X, Y, and Z groups 36, 38, and 40 may be a phosphonate (e.g., C—PO(OR)₂, where R may be an alkenyl group, an aryl group, hydrogen, or any combination thereof) or alkenyl phosphonic acid (e.g., vinyl phosphonic acid, alternative phosphonic acids with suitably functionalized double bonds in which the double bond is in conjugation with an electron withdrawing group, forming a suitable Michael acceptor, salts of phosphonic acids (e.g., sodium phosphonates or potassium phosphonates), or phosphate esters. For example, if the scale-inhibiting precursor 18 is vinyl phosphonic acid or vinyl sulfonic acid, the terminal carbon (C) atom of the alkene within vinyl phosphonic acid will bind to 34 R group of the glycerol 16 or glycerol analogue, generating the X, Y, Z group 36, 38, 40 scale-inhibiting functional group. In another embodiment, if the scale-inhibiting precursor is phosphorus oxychloride, the phosphorus (P) atom will bind or react with 34 R group of the glycerol 16 or glycerol analogue, generating a phosphate ester as a part of the X, Y, Z group 36, 38, 40 scale inhibiting functional group. In some embodiments, X, Y, Z groups 36, 38, 40 may include other functional groups (e.g., phosphates (PO₄³⁻), phosphate esters, or carboxylic acids that include one or more oxides and/or one or more hydroxides) that bind to a positive metal ion.

In some embodiments, X, Y, and Z groups 36, 38, and 40 may be the same scale-inhibiting functional group (e.g., all three X, Y, Z groups 36, 38, and 40 within the glycerol-based scale inhibitor 12 are bound to a scale-inhibiting precursor 18 such as a phosphonate ligand). In other embodiments, X, Y, and Z groups 36, 38, and 40 may include different scale-inhibiting functional groups. For example, X may be an unreacted hydroxide functional group of glycerol 16 and Y and Z may be phosphonate ligands (e.g., vinyl phosphonic acid). In another example, X and Y may be unreacted hydroxide functional groups and Z may be a phosphonate ligand (e.g., vinyl phosphonic acid). In some embodiments, a mixture of ligands may be utilized. For example, a mixture of scale-inhibiting precursor 18 may include alkenyl phosphonic acids, phosphates, and phosphorus oxychloride, leading to a glycerol-based scale inhibitor 12 having X, Y, and Z groups 36, 38, and 40 scale-inhibiting functional groups that may be a mixture of ligands, such as alkenyl phosphonic acids, phosphates, phosphate esters. In another example, X, Y, and Z groups 36, 38, and 40 scale inhibiting functional groups may be a mixture of ligands such as phosphate esters, phosphonates, and carboxylic acids. In a further example, X, Y, and Z groups 36, 38, and 40 scale inhibiting functional groups may be a mixture of ligands (e.g., phosphate groups, phosphonate groups, sulfonate groups, sulfate groups, phosphate ester groups, carboxylic acid groups, another other functional group, or a combination thereof). In general, it should be noted that the examples provided herein are meant to be non-limiting. As such, the glycerol-based scale inhibitor 12 may include advantageous properties such as tunable chelating abilities and solubility properties for use in a glycerol-based scale inhibitor solution 24 for anti-scaling applications.

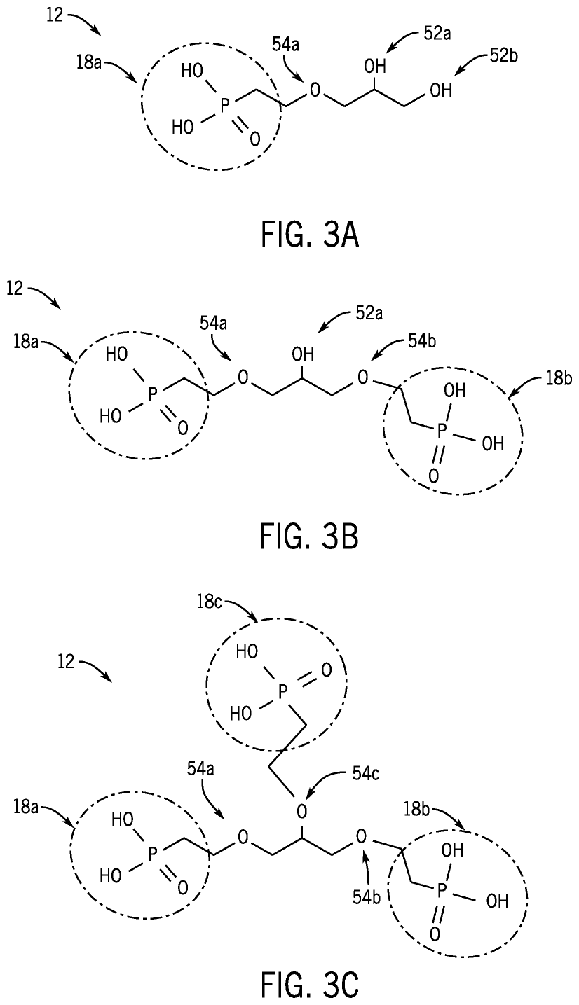

As described herein, glycerol 16 is reacted with stoichiometric equivalent amounts of the scale-inhibiting precursor 18 to generate the glycerol-based scale inhibitor 12. As referred to herein, a “stoichiometric equivalent” is an amount of a first reactant to be reacted with a second reactant such that the desired stoichiometric ratio (e.g., of the first reactant to the second reactant) is obtained. For example, in an embodiment where it is desired to obtain a glycerol-based scale inhibitor with three phosphate scale-inhibiting functional groups, three stoichiometric equivalents of a phosphate are reacted with one stoichiometric equivalent of glycerol 16. In general, the glycerol 16 includes three hydroxyl functional groups. As such, FIGS. 3A, 3B, and 3C illustrate examples of different stoichiometric equivalents of functionalization of glycerol 16 with the scale-inhibiting precursor 18 in accordance with certain embodiments of the present disclosure. Through controlled reaction conditions, various stoichiometric additions of the scale-inhibiting precursor 18 can range between one to three equivalents of the scale-inhibiting precursor 18 onto the glycerol 16.

In some embodiments, the stoichiometric ratio of the scale-inhibiting precursor 18 to the glycerol 16 may be less than 3. Accordingly, one or more of the hydroxyl functional groups of the glycerol 16 may remain unreacted (i.e., are hydroxyls) after block 14 of the method 10 of FIG. 1. For example, FIG. 3A illustrates the glycerol-based scale inhibitor 12a, where one of the hydroxyl functional groups within the glycerol molecule is functionalized with the scale-inhibiting precursor 18, as depicted within the dashed circle. The illustrated example depicts a glycerol-based scale inhibitor 12 generated with a 1:1 stoichiometric ratio of glycerol 16:scale-inhibiting precursor 18. In general, scale-inhibiting precursor 18a represents the scale-inhibiting precursor (e.g., vinyl phosphonic acid) that is bound to feature 54 (e.g. 54a, 54b, and 54c, or collectively 54), which is the O atom of the former hydroxyl functional group within glycerol 16. It should be noted that the O atom now acts as an ether in the glycerol-based scale inhibitor 12. Further, 52a and 52b (e.g., collectively 52) represent two of the three hydroxyl functional groups within glycerol 16 that remain unreacted.

Further, FIG. 3B illustrates another example of the glycerol-based scale inhibitor 12 where two hydroxyl functional groups of glycerol 16 are functionalized with two scale-inhibiting precursor 18 molecules, as illustrated within the dashed circles. The illustrated example depicts a glycerol-based scale inhibitor 12 generated with a 1:2 stoichiometric ratio of glycerol 16:scale-inhibiting precursor 18a and 18b. As such, FIG. 3B shows two vinyl phosphonic acid molecules as the scale-inhibiting precursor 18 functionalized to two of the hydroxyl functional groups within glycerol 16, while one hydroxyl group remains unreacted.

Additionally, FIG. 3C illustrates an example of the glycerol-based scale inhibitor where all three of the hydroxyl functional groups within glycerol 16 are functionalized with the scale-inhibiting precursor 18 (e.g., vinyl phosphonic acid). In particular, the illustrated example depicts a glycerol-based scale inhibitor 12 generated with a 1:3 stoichiometric ratio of glycerol 16:scale-inhibiting precursor 18a, 18b, and 18c. It should be noted that depending on the additives 22 present (e.g., other molecules, solvents, or buffers), the unreacted hydroxyl groups 52 or scale-inhibiting precursor 18 of the glycerol-based scale inhibitor 12 may or may not be protonated depending on the pH of the glycerol-based scale inhibitor solution 24. As such, the compositions described herein describe the properties of the glycerol-based scale inhibitor 12. Several non-limiting examples of the composition of the glycerol-based scale inhibitor 12 and glycerol-based scale inhibitor solution 24 are described above.

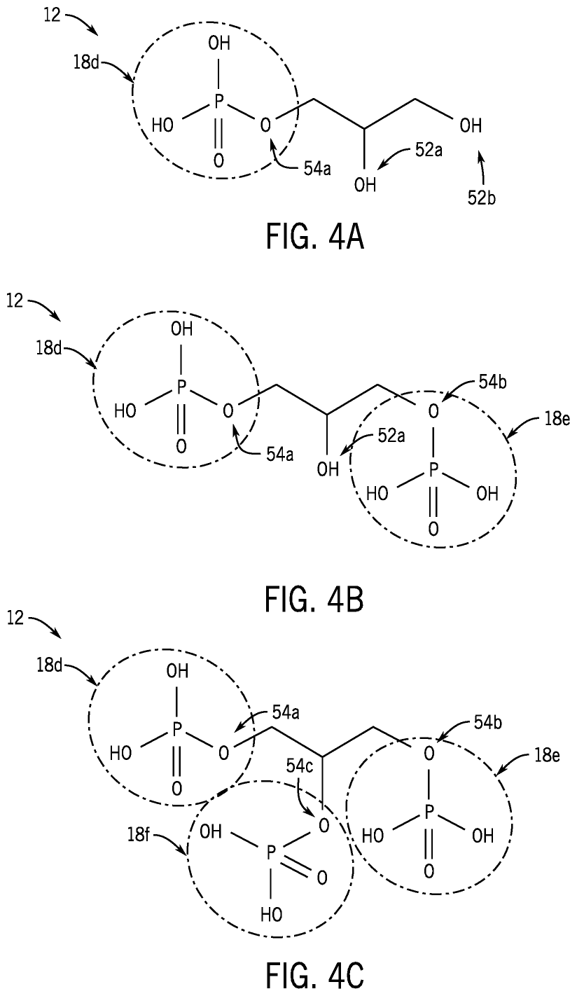

In another embodiment, the stoichiometric ratio of the scale-inhibiting precursor 18 to the glycerol 16 may be less than 3. Accordingly, one or more of the hydroxyl functional groups of the glycerol 16 may remain unreacted (i.e., are hydroxyls) after block 14 of the method 10 of FIG. 1. For example, FIG. 4A illustrates the glycerol-based scale inhibitor 12, where one of the hydroxyl functional groups within the glycerol molecule is functionalized with the scale-inhibiting precursor 18, as depicted within the dashed circle. The illustrated example depicts a glycerol-based scale inhibitor 12 generated with a 1:1 stoichiometric ratio of glycerol 16:scale-inhibiting precursor 18. In general, scale-inhibiting precursor 18d represents the scale-inhibiting functional group (e.g., phosphate esters that are formed from a reaction between scale-inhibiting precursor 18 phosphorus oxychloride and glycerol 16) that is bound to feature 54 (e.g. 54a, 54b, and 54c, or collectively 54), which is the O atom of the former hydroxyl functional group within glycerol 16. It should be noted that the O atom (feature 54) now acts as part of the phosphate ester in the glycerol-based scale inhibitor 12. Further, 52a and 52b (e.g., collectively 52) represent two of the three hydroxyl functional groups within glycerol 16 that remain unreacted.

Further, FIG. 4B illustrates another example of the glycerol-based scale inhibitor 12 where two hydroxyl functional groups of glycerol 16 are functionalized with two scale-inhibiting precursor 18 molecules, as illustrated within the dashed circles. The illustrated example depicts a glycerol-based scale inhibitor 12 generated with a 1:2 stoichiometric ratio of glycerol 16:scale-inhibiting precursor 18d and 18e. As such, FIG. 4B shows two phosphate ester molecules as the scale-inhibiting precursor 18 functionalized to two of the hydroxyl functional groups within glycerol 16, while one hydroxyl group remains unreacted.

Additionally, FIG. 4C illustrates an example of the glycerol-based scale inhibitor where all three of the hydroxyl functional groups within glycerol 16 are functionalized with the scale-inhibiting precursor 18 (e.g., phosphate ester). In particular, the illustrated example depicts a glycerol-based scale inhibitor 12 generated with a 1:3 stoichiometric ratio of glycerol 16:scale-inhibiting precursor 18d, 18e, and 18f. It should be noted that depending on the additives 22 present (e.g., other molecules, solvents, or buffers), the unreacted hydroxyl groups 52 or scale-inhibiting precursor 18 of the glycerol-based scale inhibitor 12 may or may not be protonated depending on the pH of the glycerol-based scale inhibitor solution 24. As such, the compositions described herein describe the properties of the glycerol-based scale inhibitor 12. Several non-limiting examples of the composition of the glycerol-based scale inhibitor 12 and glycerol-based scale inhibitor solution 24 are described above.

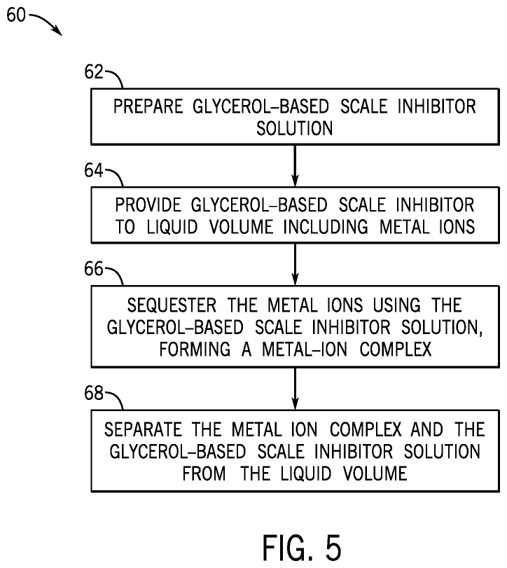

In general, the glycerol-based scale inhibitor 12 can be utilized to prepare a glycerol-based scale inhibitor solution 24 for subsequent use in mitigating inorganic scale in industrial applications. With the preceding in mind, FIG. 5 illustrates a flow diagram of a method 60 for using the glycerol-based scale inhibitor solution 24 for sequestering metal ions in accordance with certain embodiments of the present disclosure. In general, the glycerol-based scale inhibitor solution 24 may be used in anti-scaling applications. At block 62, the method 60 includes preparing the glycerol-based scale inhibitor solution 24. In general, block 62 may be performed in a generally similar manner as described in FIG. 1. As such, a glycerol-based scale inhibitor solution 24 is provided to a liquid volume that includes metal ions, as described at block 64. The addition of the glycerol-based scale inhibitor solution 24 sequesters (i.e., chelates) the metal ions, thereby forming a metal-ion complex. The metal-ion complex and the glycerol-based scale inhibitor solution 24 are subsequently separated from the liquid volume. As described herein, the glycerol-based scale inhibitor solution 24 may include advantageous properties, such as tunable chelating ability and solubility properties in liquid volumes for sequestering or chelating to metal ions.

At block 64, the method 60 provides a glycerol-based scale inhibitor solution 24 to a liquid volume that includes metal ions. The glycerol-based scale inhibitor solution 24 includes glycerol-based scale inhibitor 12 and additives 22. It is presently recognized that the additives 22 may include, but are not limited to, other molecules, solvents, or buffers. As such, the additives 22 may include buffering agents, such as phosphate-based buffers, hydroxide-based buffers, carbonate-based buffers, bicarbonate-based buffers, amine-based buffers, acetate-based buffers, or any combination thereof. Further, solvents may be provided as an additive 22 during the production of the glycerol-based scale inhibitor solution 24. The solvent may serve as a reaction medium and may phosphonates, phosphate esters, sulfonates, sulfates, phosphates, alkenyl phosphonic acids, glycols, water, carboxylic acids, alcohols, protic solvents, solvents miscible with water, or a combination thereof. The glycerol-based scale inhibitor solution 24 may be provided as a liquid to a liquid volume. In another embodiment, the glycerol-based scale inhibitor solution 24 may be provided to a liquid volume containing solids. In general, the glycerol-based scale inhibitor solution 24 may be miscible or immiscible with the liquid volume.

Referring to the method 60, at block 66, after the glycerol-based scale inhibitor solution 24 is provided to a liquid volume including metal ions, the glycerol-based scale inhibitor solution 24 may sequester, bind, or chelate to metal ions in solution, forming a metal-ion complex. For example, the glycerol-based scale inhibitor may bind to metal carbonates (e.g., wherein the metal may be calcium, iron, or any other metal thereof), metal sulfates (e.g., wherein the metal may be calcium, iron, or any other metal thereof), metal hydroxides (e.g., wherein the metal may be calcium, iron, or any other metal thereof), metal sulfides (e.g., wherein the metal may be calcium, iron, or any other metal thereof), zinc sulfides, lead sulfides, or any combination thereof. At block 68, the metal-ion complex and the glycerol-based scale inhibitor solution 24 are separated from the liquid volume. Separation techniques include, but are not limited to, acid-base extraction, centrifugation, filtration, etc. As such, this method allows for sequestration and separation of inorganic scale from industrial applications.

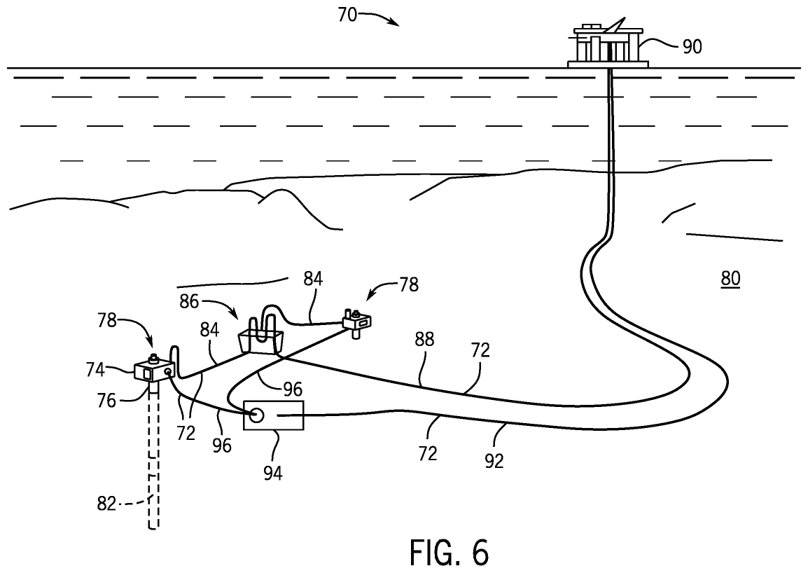

With the foregoing in mind, FIG. 6 illustrate a schematic view of a subsea system 70 where the glycerol-based scale inhibitor solution 24 may be employed in mitigating inorganic scale in oil and gas production. In general, the subsea system 70 includes electrical cables 72 used for transmitting information and primary electrical power for various subsea components (e.g., actuators, sensors, etc.). The subsea system 70 may include a subsea hydrocarbon production system configured to extract oil or gas from a subterranean reservoir, a subsea fluid injection system configured to inject fluid (e.g., liquid or gas) into a subterranean reservoir, or any other subsea system associated with subterranean reservoirs. For example, the subsea fluid injection system may include a subsea gas, water, glycerol-based scale inhibitor solution 24 for mitigating inorganic scale. In certain embodiments, the subsea system 70 may include a subsea tree 74 coupled to a wellhead 76 to form a subsea station 78 configured to extract and/or inject fluids relative to a subterranean reservoir. For example, the subsea station 78 may be configured to extract formation fluid, such as oil and/or natural gas, from the sea floor 80 through the well 82. By further example, the subsea station 78 may be configured to inject CO₂ into the subterranean reservoir. In some embodiments, the subsea system 70 may include multiple subsea stations 78 that extract and/or inject fluids relative to respective wells 82.

In certain embodiments of the subsea system 70 configured for production, after passing through the subsea tree 74, the formation fluid flows through fluid conduits or pipes 84 to a pipeline manifold 86. The pipeline manifold 86 may connect to one or more flowlines 88 to enable the formation fluid to flow from the wells 82 to a surface platform 90. In some embodiments, the surface platform 90 may include a floating production, storage, and offloading unit (FPSO) or a shore-based facility. In addition to flowlines 88 that carry the formation fluid away from the wells 82, the subsea system 70 may include lines or conduits 92 that supply fluids, as well as carry control and data lines to the subsea equipment. These conduits 92 connect to a distribution module 94, which in turn couples to the subsea stations 78 via supply lines 96. In some scenarios, the surface platform 90 may be located a significant distance (e.g., greater than 100 m, greater than 1 km, greater than 10 km, or greater than 60 km) away from the wells 82. As discussed in further detail below, the subsea system 70 (e.g., the subsea tree 74, the subsea station 78, the pipeline manifold 86, and/or the distribution module 94) may include a subsea power system (e.g., subsea power bus system) that provides secondary power from energy storage units (e.g., batteries, fuel cells, or super capacitors (for initial actuator movement)) over one or more buses to various subsea components (e.g., actuators, sensors, etc.). For example, the subsea power system may be configured to provide secondary power, such as during a power loss from the primary power from the electrical cables 72, to operate various valves, sensors, and other subsea components. While the subsea system described above is for extracting hydrocarbons, it should be understood that the present disclosure may also apply to other types of subsea systems 70, such as subsea injection systems (e.g., subsea gas injection system, subsea water injection system, subsea carbon dioxide injection system).

Accordingly, the present disclosure is directed to techniques for generating a glycerol-based scale inhibitor using glycerol and a scale-inhibiting precursor and further adding additives to generate a glycerol-based scale inhibitor solution. In this way, a glycerol-based scale inhibitor in the presence of an additive feed may generate a glycerol-based scale inhibitor solution having certain chemical compositions, such as different stoichiometric degrees of functionalization, type of functional group, length of main chain carbon, and pH-mediated binding availability that are useful for mitigating inorganic scale. For example, the resulting glycerol-based scale inhibitor solution may be provided to a liquid volume including metal ions, forming a metal ion complex to reduce inorganic scale. As described above, the disclosed glycerol-based scale inhibitor solution may be used in direct anti-scaling applications due to the different stoichiometric degrees of functionalization, type of functional group, length of main chain carbon, and pH-mediated binding availability. In any case, the glycerol-based scale inhibitor solution may be utilized in oil and gas, water treatment, power plant, or geothermal applications.

The technical effect of the disclosed embodiments includes a glycerol-based scale inhibitor with tunable chelating activity by adjusting the stoichiometric ratio of scale-inhibiting precursors. Additionally, or alternatively, the chelating activity of the glycerol-based scale inhibitor may be tuned through the use of different scale-inhibiting precursors. For example, the glycerol-based scale inhibitor may include one or multiple different scale-inhibiting functional groups. The chelating activity of the glycerol-based scale inhibitor solution may also be tuned by the use of additives, such as buffers, corrosive inhibitors, and the like.

The subject matter described in detail above may be defined by one or more clauses, as set forth below.

A method includes providing a glycerol feed and providing a scale-inhibiting precursor feed. The method also includes generating one or more glycerol-based scale inhibitors by contacting the glycerol feed with the scale-inhibiting precursor feed. The method also includes providing one or more additives to the glycerol-based scale inhibitor, thereby generating a glycerol-based scale inhibitor solution.

The method of the preceding clause, wherein the glycerol-based scale inhibitor solution comprises the one or more glycerol-based scale inhibitors and the one or more additives.

The method of any preceding clause, wherein the scale-inhibiting precursor feed comprises alkenyl phosphonic acids.

The method of any preceding clause, wherein the scale-inhibiting precursor feed comprises phosphate esters.

The method of any preceding clause, wherein the scale-inhibiting precursor comprises functionalized carboxylic acids.

The method of any preceding clause, wherein the scale-inhibiting precursor comprises sulfonates.

The method of any preceding clause, wherein the scale-inhibiting precursor comprises a combination of alkenyl phosphonic acids, phosphate esters, sulfonates, and carboxylic acids.

The method of any preceding clause, wherein the one or more additives comprise phosphate-based buffers, hydroxide-based buffers, carbonate-based buffers, bicarbonate-based buffers, amine-based buffers, acetate-based buffers, or any combination thereof.

A composition includes a glycerol analogue core and one or more scale-inhibiting functional groups linked to the glycerol analogue core via one or more ethers of the glycerol analogue core.

The composition of the preceding clause, wherein the glycerol analogue core comprises three carbon atoms.

The composition of any preceding clause, wherein the one or more scale-inhibiting functional groups comprise alkenyl phosphonic acids.

The composition of any preceding clause, wherein the one or more scale-inhibiting functional groups comprise phosphates.

The composition of any preceding clause, wherein the one or more scale-inhibiting functional groups comprise sulfonates.

The composition of any preceding clause, wherein the one or more scale-inhibiting functional groups comprise a mixture of phosphates and alkenyl phosphonic acids.

The composition of any preceding clause, wherein the one or more scale-inhibiting functional groups are a combination of two or more of phosphates, alkenyl phosphonic acids, sulfonates, and phosphate esters.

The composition of any preceding clause, wherein the glycerol analogue core comprises less than three hydroxyl functional groups.

The composition of any preceding clause, further comprising one or more buffers and one or more solvents.

A method includes providing a glycerol-based scale inhibitor solution. The method also includes providing a liquid volume, wherein the liquid volume comprises metal ions. The method also includes sequestering the metal ions with the glycerol-based scale inhibitor solution, thereby forming a metal-ion complex. The method also includes separating the metal-ion complex and the glycerol-based scale inhibitor solution from the liquid volume.

The method of preceding clause, wherein the metal ions comprise metal carbonates, metal sulfates, metal hydroxides, metal sulfides, zinc sulfides, lead sulfides, or any combination thereof.

The method of any preceding clause, wherein the glycerol-based scale inhibitor solution is injected via a subsea fluid injection system into a subterranean reservoir to mitigate inorganic scale.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principals of the disclosure and its practical applications, to thereby enable others skilled in the art to best utilize the disclosure and various embodiments with various modifications as are suited to the particular use contemplated.

Finally, the techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112 (f). However, for any claims containing elements designated in any other manner, it is intended that such elements are not to be interpreted under 35 U.S.C. 112 (f).

1. A method comprising:
providing a glycerol feed;
providing a scale-inhibiting precursor;
generating one or more glycerol-based scale inhibitors by contacting the glycerol feed with the scale-inhibiting precursor; and
providing one or more additives to the one or more glycerol-based scale inhibitors, thereby generating a glycerol-based scale inhibitor solution.

2. The method of claim 1, wherein the glycerol-based scale inhibitor solution comprises the one or more glycerol-based scale inhibitors and the one or more additives.

3. The method of claim 1, wherein the scale-inhibiting precursor comprises alkenyl phosphonic acids.

4. The method of claim 1, wherein the scale-inhibiting precursor comprises phosphate esters.

5. The method of claim 1, wherein the scale-inhibiting precursor comprises functionalized carboxylic acids.

6. The method of claim 1, wherein the scale-inhibiting precursor comprises sulfonates.

7. The method of claim 1, wherein the scale-inhibiting precursor comprises a combination of alkenyl phosphonic acids, phosphate esters, sulfonates, and carboxylic acids.

8. The method of claim 7, wherein the one or more additives comprise phosphate-based buffers, hydroxide-based buffers, carbonate-based buffers, bicarbonate-based buffers, amine-based buffers, acetate-based buffers, or any combination thereof.

9. A composition comprising:
a glycerol analogue core; and
one or more scale-inhibiting functional groups linked to the glycerol analogue core via one or more ethers of the glycerol analogue core.

10. The composition of claim 9, wherein the glycerol analogue core comprises three carbon atoms.

11. The composition of claim 9, wherein the one or more scale-inhibiting functional groups comprise alkenyl phosphonic acids.

12. The composition of claim 9, wherein the one or more scale-inhibiting functional groups comprise phosphates.

13. The composition of claim 9, wherein the one or more scale-inhibiting functional groups comprise sulfonates.

14. The composition of claim 9, wherein the one or more scale-inhibiting functional groups comprise a mixture of phosphates and alkenyl phosphonic acids.

15. The composition of claim 9, wherein the one or more scale-inhibiting functional groups are a combination of two or more of phosphates, alkenyl phosphonic acids, sulfonates, and phosphate esters.

16. The composition of claim 9, wherein the glycerol analogue core comprises less than three hydroxyl functional groups.

17. The composition of claim 9, further comprising one or more buffers and one or more solvents.

18. A method comprising:
providing a glycerol-based scale inhibitor solution;
providing a liquid volume comprising metal ions;
sequestering the metal ions with the glycerol-based scale inhibitor solution, thereby forming a metal-ion complex; and
separating the metal-ion complex and the glycerol-based scale inhibitor solution from the liquid volume.

19. The method of claim 18, wherein the metal ions comprise metal carbonates, metal sulfates, metal hydroxides, metal sulfides, zinc sulfides, lead sulfides, or any combination thereof.

20. The method of claim 18, wherein the glycerol-based scale inhibitor solution is used to mitigate inorganic scale in oil and gas systems, geothermal applications, and industrial water treatment plants.
